(19) United States
(12) Patent Application Publication
(10) Pub. No.: US 2011/0097386 A1
(43) Pub. Date: Apr. 28, 2011

(54) STABILIZED GLUCAGON SOLUTIONS

(75) Inventors: Solomon S. Steiner, Mount Kisco, NY (US); Robert Hauser, Columbia, MD (US); Ming Li, Yorktown Heights, NY (US); Robert Feldstein, Yonkers, NY (US); Roderike Pohl, Sherman, CT (US)

(73) Assignee: Biodel, Inc.

(21) Appl. No.: 12/715,203

(22) Filed: Mar. 1, 2010

Related U.S. Application Data
(60) Provisional application No. 61/254,128, filed on Oct. 22, 2009.

(51) Int. Cl. A61K 38/26; A61K 9/127; A61P 3/08

(52) U.S. Cl. 424/450; 514/11.7

(57) ABSTRACT

A formulation composed of a sugar such as glucose and a surfactant such as myristoyl lysophosphocholine (LMPC) has been designed to stabilize both hydrophilic and hydrophobic portions of the glucagon molecule, under prolonged physiological conditions, in a formulation that is sufficiently similar to the pH and osmolarity of plasma so as not to induce or to minimize site irritation. The combination of a simple sugar and an surfactant stabilizes the glucagon molecule in an aqueous solution for seven days at 37° C.

### CROSS-REFERENCE TO RELATED APPLICATIONS

This application claims benefit of and priority to U.S. Ser. No. 61/254,128 filed on Oct. 22, 2009, which is incorporated herein by reference.

### FIELD OF THE INVENTION

This disclosure generally relates to stabilized glucagon solutions.

### BACKGROUND OF THE INVENTION

Glucagon is synthesized in the pancreas. It is a highly conserved polypeptide consisting of a single chain of 29 amino acids, with a molecular weight of 3485 Da. Recombinant glucagon is expressed in E. coli and purified to at least 98% pure prior to use. Proteolytic removal of the amino-terminal histidine residue leads to loss of the biological activity. Glucagon has a helical conformation in the crystalline state, while in dilute aqueous solutions it has a random coil conformation with 15% alpha helix at the C-terminal end.

Pharmacologically, glucagon increases the concentration of glucose in the blood. The first six amino acids at the N-terminus of the glucagon molecule bind to specific receptors on liver cells. This leads to an increase in the production of cAMP, which facilitates the catabolism of stored glycogen and increases hepatic gluconeogenesis and ketogenesis. The immediate pharmacologic result is an increase in blood glucose at the expense of stored hepatic glycogen. The onset of action post injection is 5-20 minutes. Glucagon is degraded in the liver, kidney, and tissue receptor sites. The half life of glucagon in plasma is 3 to 6 minutes, similar to that of insulin.

Glucagon is soluble in aqueous solutions at pH less than 3 or greater than 9, and has low solubility in the pH range of 4 to 8 due to its isoelectric point of 7.1. It readily forms a gel in acidic aqueous conditions (pH 3-4) and precipitates within an hour of preparation in a neutral aqueous solution.

Currently, the commercial preparation of glucagon is a two part sterile vial, intended for immediate use following reconstitution. It is sold as a rescue kit and is available for intravenous, intramuscular or subcutaneous administration. The kit contains 1 mg (1 unit) of glucagon and 49 mg of lactose in a sterile vial. The diluent contains 12 mg/mL glycerin, water for injection and hydrochloric acid. The diluent is injected into the powder vial, gently swirled to dissolve the glucagon, then the glucagon solution is pulled back into the same syringe ready for injection. The pH of this solution is approximately 2. The recommended dose is typically 0.5-1 mg. Any reconstituted glucagon is to be discarded since it is not stable in solution.

Previous attempts to stabilize glucagon include the addition of cationic or anionic monovalent detergents to enhance the solubilization of 1 mg/mL glucagon using a 6 fold molar excess of detergent, as described in GB Patent No. 1202607; hen egg lysolecithin, which shows the detergent induced partial helical structure in solutions of glucagon containing about 0.02 mg/ml peptide, as described in J. Biol. Chem 247, 4986-4991; 4992-4996 (1972); lysolecithin, as described in Biopolymers 21, 1217-1228 (1982), Biopolymers 22, 1003-1021 (1983); micelles of anionic detergent SDS at low pH, as described in Biochem. 19, 2117-2122 (1980), and at neutral pH, as described in Biochim. Biophys. Acta 603, 298-312 (1980); and cyclodextrins (J. Pharm Sci. 97(7):2720-9 (2008); Eur J Pharm Sci. 2; 36(4-5):412-20 (2009). EP 1061947 by Novo Nordisk describes stablized glucagon solutions containing surfactant such as LPMC or other detergents carrying multiple charges (two or more negative, two or more positive, or both positive and negative) added in 0.5-20 moles detergent/peptide), solubilizing glucagon at pharmaceutically relevant concentrations in the entire pH range of 4 to 9. U.S. Pat. No. 5,652,216 to Kornfelt, et al., describes a pharmaceutical preparation comprising glucagon and a stabilizing amount of a pharmaceutically acceptable ampholyte such as an amino acid or dipeptide or a mixture thereof and optionally an excipient.

Recently, glucagon is being developed for use in an “artificial pancreas” or bihormonal pump. Insulin pumps have been used by insulin dependent diabetics for over a decade. These pumps are capable of providing a continuous flow of insulin to cater to their basal insulin needs. After eating, the user can manually increase the insulin flow to temporarily cover their meal, then cut back to the slow basal flow. These apparatus are attached to the abdominal surface by a small needle and may remain in place for up to a week. Newer devices also have been developed that combine the ability to read the patient glucose levels and deliver insulin as needed to cover individual patients requirements. However, should too much insulin be given, there is no way to prevent hypoglycemia. Therefore, the next step to complete the artificial pancreas is to add a second pump to deliver glucagon to the patient to counteract hypoglycemia. This creates an artificial pancreas capable of keeping a patient within ideal glucose levels, similar to how a normal functioning pancreas does in a non-diabetic individual. However, this application requires a glucagon that is stable in solution for at least seven days at 30-37° C., and the current commercial formulations are not capable of fulfilling that need. Moreover, since the currently available formulation is designed for “rescue” use, the acidic nature and pain of the injection is acceptable since it is a single dose, rarely given to the patient. However, the pH and isotonicity of the solution should be closer to physiological conditions for use in a pump.

It is therefore an object of the present invention to provide a glucagon that is stable as a clear solution for at least seven days at 37° C. for extended use in a pump device.

### SUMMARY OF THE INVENTION

A diluent composed of a sugar such as glucose and a surfactant such as myristoyl lysophosphocholine (LMPC) has been designed to stabilize both hydrophilic and hydrophobic portions of the glucagon molecule, under prolonged physiological conditions, in a formulation that is sufficiently similar to the pH and osmolarity of plasma to minimize site irritation. In the preferred embodiment, the sugar is glucose which can assist in the elevation of blood sugar on injection. The combination of a simple sugar and an amphiphilic surfactant stabilizes the glucagon molecule in an aqueous solution for at least seven days at 37° C. The surfactant is believed to induce a helical structure in the hydrophobic portions of the glucagon, and the simple sugars are believed to stabilize the hydrophilic regions of the polypeptide. The combination stabilizes the glucagon at a concentration of 1 mg/mL at physiological osmolarity and pH. Additional exicipients may be added to stabilize the formulation or control gelation or viscosity. The formulation may also be in the form of a microemulsion or liposomes, although this embodiment is not for use with a pump or small gauge needle.

In the preferred embodiment shown in the examples, the stabilized glucagon solution contains water, lyso myristoyl phosphocholine (LMPC), glucose and a preservative such as sodium benzoate. The concentration range for glucagon is 0.5-5 mg/mL, preferably 0.8 to 1.5 mg/mL; glucose 20-100 mg/mL, preferably 36 to 72 mg/mL, LMPC 0.1-10 mg/mL, preferably 0.5-5 mg/mL; preservative sodium benzoate or benzyl alcohol 0.2 to 3 mg/mL.

In the preferred embodiment, the product may be produced and stored at 4° C. as a clear, one part solution, ready for injection (subcutaneously, intramusculary, or intravenously).

In another embodiment, the glucagon is lyophilized in the presence of glucose and surfactant (preferably LMPC), to stabilize the powder, and on reconstitution assist in stabilizing the glucagon in solution. The diluent may contain a preservative, such as sodium benzoate, benzyl alcohol or m-cresol. This system works as a two part diluent and dry powder system that is stable at room temperature. On reconstitution of the powder with the diluent, the resulting clear solution may be used up to 7 days next to the body at a temperature of 30-37° C., for example, in an insulin pump.

### BRIEF DESCRIPTION OF THE DRAWINGS

FIG. 1 is a schematic of the structure of glucagon.

FIG. 2 is a graph of percent remaining glucagon over time (days) at 25° C. at pH 4.7: control HCl (diamond), 0.6M glucose (solid square), 0.3M glucose (empty square), and 0.3M sucrose (-x-).

FIG. 3 is a graph of percent glucose in solution over time (days) at 37° C. Comparison of LMPC alone (star), LMPC+glucose (diamond), LMPC+lactose (square) and LMPC+glycerin (open circle).

FIG. 4 is a graph of percent glucagon in solution over time (days) at 37° C. Accelerated stability study of BIOD 901 (glucagon 1 mg/mL+2 mg/ml LMPC+45 mg/mL glucose+2 mg/mL m-cresol, Diamond) compared to Lilly glucagon at pH 2 (open triangle) and pH 4 (solid triangle).

FIG. 5 is a graph of percent glucagon in solution over time (days) at 37° C., comparing BIOD 901 (glucagon 1 mg/mL+2 mg/ml LMPC+45 mg/mL glucose+2 mg/mL m-cresol, diamonds) compared to Lilly glucagon at pH 2 (open triangles).

FIG. 6 is a graph of percent glucagon over time (days) at 37° C., comparing two preservatives sodium benzoate (open diamonds) and m-cresol (closed diamonds) in glucagon formulation BIOD 901.

FIG. 7 is a graph of the percent glucagon in solution over time (days) at 37° C., comparing BIOD 901 (diamonds) and 902 (circles).

FIG. 8 is a graph of percent glucagon over time (days) at 37° C., comparing the effect of removing glucose (star), replacing glucose with lactose (solid square), adding EDTA (empty circle) or removing glucose and adding EDTA and lactose (empty square) to BIOD 902 (solid circle).

FIG. 9 is a graph of the corrected baseline of glucose values over time following glucagon administration to miniature diabetic swine, comparing Lilly glucagon pH 2 (open triangles) versus Biodel glucagon (BIOD 901, solid diamonds).

FIG. 10 is a graph of the corrected baseline of glucose values over time (days) following glucagon administration to miniature diabetic swine, comparing BIOD 901 freshly prepared (solid diamonds) to a sample that was incubated 3 days at 37° C. (open diamonds).

### DETAILED DESCRIPTION OF THE INVENTION

### I. Definitions

As used herein, “glucagon” refers to the full length peptide, glucagon. “GLP-1” refers to glucagon-like peptides (GLP-1, amino acids 7-36 amide and 7-37), and analogs and derivatives thereof, unless otherwise specified.

As used herein, a “sugar” refers to a monosaccharide or disaccharide, small organic molecules that contain multiple hydroxy groups and an aldehyde or ketone functional group. Saccharides can exist in both a straight chain or cyclic conformation. Preferred examples include sucrose, maltose and glucose.

As used herein, “osmolarity” is the concentration of a solution in terms of milliosmoles of solutes per liter of solution. The normal plasma osmolarity is in the range of 280-310 mOs/kg.

As used herein, “prolonged” refers to a period of five to ten days, preferably seven to ten days.

As used herein, “physiological pH” is in the range of 6.8 to 7.5, preferably 7 to 7.4.

As used herein, “physiological temperature” is between 30 and 37° C.

### II. Formulations

A. Glucagon

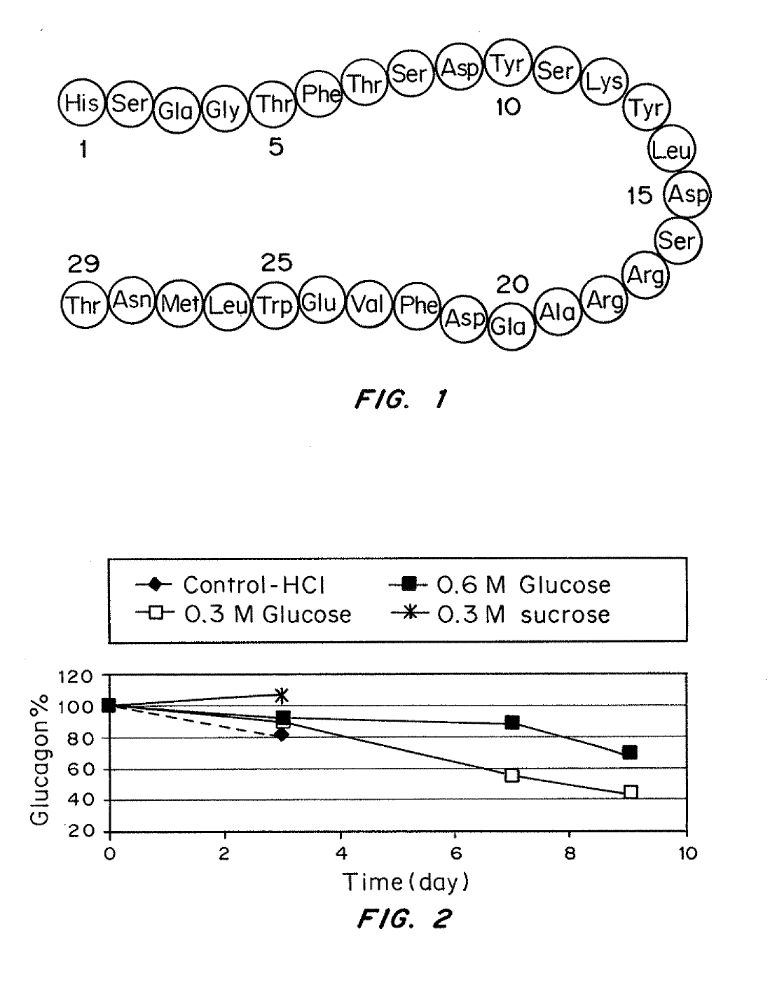

Glucagon is a highly conserved polypeptide consisting of a single chain of 29 amino acids (FIG. 1), with a molecular weight of 3485 Da, synthesized in the pancreas. Recombinant glucagon is expressed in E. coli and purified to at least 95% pure prior to use. Natural and recombinant glucagon are bioequivalent, as demonstrated by Graf, et al., J. Pharm. Sci. 88(10):991-995 (2000). Multiple commercial sources are available. The preferred concentration range for glucagon is 0.5-5 mg/mL, preferably 0.8 to 1.5 mg/mL, most preferably 1 mg/mL.

B. Sugars

“Sugar” refers to a monosaccharide or disaccharide, small organic molecules that contain multiple hydroxy groups and an aldehyde or ketone functional group, but not polyols such as glycerol. Saccharides can exist in both a straight chain or cyclic conformation. Preferred examples include sucrose, maltose and glucose in a concentration range of about 20-100 mg/mL, preferably 0.25 M.

C. Surfactants

Amphiphilic surfactants (i.e., having at least two positive and two negative charges in different regions of the molecule) such as phospholipids or glycerophospholipids, containing a polar head and two non-polar tails, in combination with sugars are useful in stabilizing the glucagon. These are preferably GRAS (“generally regarded as safe”) phospholipids or endogenous phospholipids. The surfactant may be a sn-glycero-3-phosphate ester of ethanolamine, choline, serine or threonine. Octanoyl, decanoyl, lauroyl, palmitoyl and myristoyl derivatives of lysophosphatidylcholine, lysophosphatidylserine and lysophosphatidylthreonine, are particularly useful.

In the preferred embodiment, the surfactant is LMPC. Surfactant is added in a concentration equivalent to LMPC in a range of 0, 1-10 mg/mL, preferably 0.5-5 mg/mL. A preferred concentration is 2 mg surfactant/mL with glucose at 0.25 M.

Surfactant may interact with the glucagon solution to form liposomes. Liposomes (LPs) are spherical vesicles, composed of concentric phospholipid bilayers separated by aqueous compartments. LPs have the characteristics of adhesion to and creating a molecular film on cellular surfaces. Liposomes are lipid vesicles composed of concentric phospholipid bilayers which enclose an aqueous interior (Gregoriadis, et al., Int J Pharm 300, 125-30 2005; Gregoriadis and Ryman, Biochem 3 124, 58P (1971)). The lipid vesicles comprise either one or several aqueous compartments delineated by either one (unilamellar) or several (multilamellar) phospholipid bilayers (Sapra, et al., Curr Drug Deliv 2, 369-81 (2005)). The success of liposomes in the clinic has been attributed to the nontoxic nature of the lipids used in their formulation.

Liposomes have been widely studied as drug carriers for a variety of chemotherapeutic agents (approximately 25,000 scientific articles have been published on the subject) (Gregoriadis, N Engl J Med 295, 765-70 (1976); Gregoriadis, et al., Int J Pharm 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadl, Curr Med Chem 12, 2193-214 (2005); Tyagi, et al., J Urol 171, 483-9 (2004)).

The formulation can also be provided as an emulsion, microemulsion (<100 nm) or micelles, formed by addition or water to the surfactant, or surfactant to the water. These embodiments are not preferred for use with a pump or other small orifice means for administration, due to the inherently more viscous nature of liposomes and emulsions.

Non-ionic surfactants such as methyl beta cyclodextran or polysorbates (such as TWEEN 20) also may be used to control gelation of the above excipients and/or glucagon.

D. Optional Excipients: Preservatives

Preservatives such as EDTA, sodium benzoate, metacresol, and benzyl alcohol may be added to the formulation to a concentration of 0.2 to 3 mg/mL. The preservative may be present in the liquid formulation, or in a diluent for the two part lyophilized presentation.

Excipients may also be added to adjust osmolarity. For example, glycerol, in a final concentration of 15-22 mg/mL may be used to adjust osmolarity.

### II. Methods of Reconstitution and Use

In the preferred embodiment, the product is a clear one part solution, stored at 4° C. ready for injection. In the preferred embodiment shown in the examples, the stabilized glucagon solution contains water, lyso myristoyl phosphocholine (LMPC), glucose and sodium benzoate. The concentration range for glucagon is 0.5-5 mg/mL, preferably 0.8 to 1.5 mg/mL; glucose 20-100 mg/mL, preferably 36 to 72 mg/mL, LMPC 0.1-10 mg/mL, preferably 0.5-5 mg/mL; preservative sodium benzoate or benzyl alcohol 0.2 to 3 mg/mL.

In one embodiment, the glucagon is lyophilized in the presence of glucose and surfactant, preferably LMPC, to stabilize the powder, and on reconstitution assists in stabilizing the glucagon in solution. The diluent may contain a preservative, preferably sodium benzoate, benzyl alcohol or m-cresol. This system works as a two part diluent and dry powder system that is stable at room temperature. On reconstitution of the powder with the diluent, the resulting clear solution may be used up to 7 days next to the body at a temperature of 30-37° C.

Final pH of the reconstituted solution is in the range of 4-8, preferably 5-7.6. Osmolarity in the range of 200-600 mOsm, preferably 290-310 mOsm.

To use the glucagon in a pump, pump cartridges are prefilled. It may also be produced as a kit containing two injection vials, one containing a dry sterile powder glucagon and the other a sterile diluent. The volume of both vials is 1 to 5 mL, depending on the volume to be dispensed by the pump device. At the time of use, the contents of the diluent vial are added to the glucagon vial via a transfer syringe and gently swirled to reconstitute. Then a 1.5 to 3 mL syringe is filled, for example, using a needle inserted into sterile vial, with the clear glucagon solution, and is placed directly in the pump device, after removal of the needle. Alternatively, the needle/syringe may be used to fill a resevoir provided by the pump manufacturer which is then inserted into or as part of the device. At the end of 5 days, the remaining glucagon solution is discarded and fresh reconstituted glucagon solution is provided to the pump. The dose of glucagon delivered to the subcutaneous tissue will be determined by the needs of the patient. A typical dose used to reverse severe hypoglycemic events is 1 mL of a 1 mg/mL solution.

The present invention will be further understood by reference to the following non-limiting examples.

### Example 1

### Simple Sugars for Glucagon Stabilization

This initial study was designed to compare glucagon stabilization with sucrose and glucose at different concentrations at pH 4.7, 25° C. Glucagon solutions were prepared to a concentration of approximately 1 mg/mL and mixed with either (1) HCl (control), (2) 0.6M glucose, (3) 0.3M glucose, or (4) 0.3M sucrose.

Although the sucrose-stabilized glucagon was stable at day 3, it gelled at day 4. The control in HCl also rapidly degraded and gelled at day 4. 0.6 M glucose was effective to maintain the glucagon at 90% of original 1 mg/mL dose for 7 days (FIG. 2). A similar result was seen at pH 3.6 and over the temperature range of 25-37° C.

Glucose alone is somewhat effective at stabilizing glucagon. However, the higher concentration 0.6M (hypertonic) is better than 0.3M (physiologic). The hypertonic solution is likely to create injection site reactions. It is desirable to formulate at a higher pH, but the addition of sugar alone is limited to pH 4.7 to due to the limited solubility of glucagon approaching the isoelectric point. Therefore, it is preferable to find another stabilizing or solubilizing agent to work in combination with glucose to increase the solubility at higher pH and lengthen the duration of stability at 37° C.

### Example 2

### Studies Showing the Effect of Different Sugars on the Stability of Glucagon in Combination with LMPC

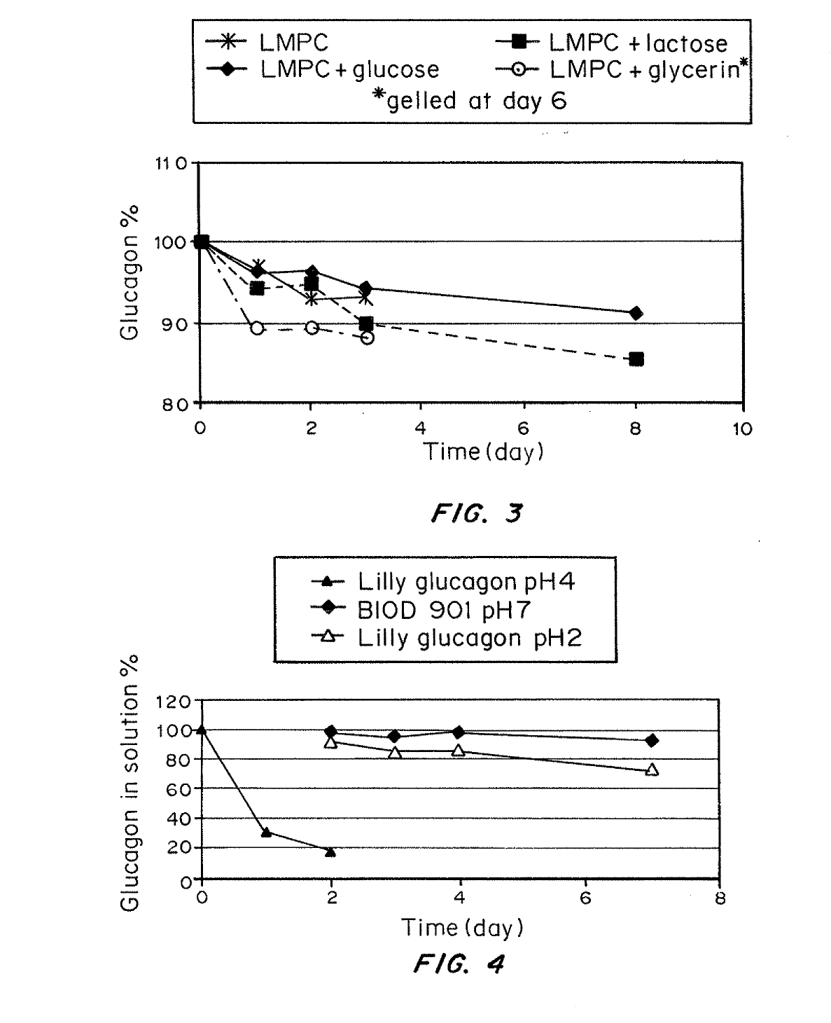

To further optimize the glucagon formulation, LMPC was added to increase the solubility of glucagon at neutral pH. The glucagon LMPC was formulated with several sugars to determine whether the formulation stability could be extended beyond the original glucagon/glucose formulation (FIG. 2). The sugars selected were lactose (90 mg/mL), glucose (45 mg/mL) and glycerin (23 mg/mL). The test sugar+LMPC formulations were compared to LMPC (2 mg/mL) alone following incubation at 37° C. The results are shown in FIG. 3.

The results of this study found that glucagon with LMPC+glycerin and glucagon+LMPC alone gelled by day 6. Lactose and glucose remained in solution to day 8, however, though these were not observed to gel, the glucose more effectively chemically stabilized glucagon than did lactose. Therefore, glucose in conjunction with LMPC is the preferred combination for glucagon stabilization.

### Example 3

### Development of a Stable Glucagon Formulation for Use in Bihormonal Pumps

The purpose of this example was to make a stable glucagon suitable for use with a bihormonal pump (artificial pancreas). For this purpose, an antibacterial agent or preservative is added to complete the formulation. Adequate physical stability at 37° C. is also required, since the pump is close to the body, exposing it to physiological temperatures. The tubing of the pump must also be free of any particulate matter, gels or fibrils for at least 5 days at 37° C. for the pump to accurately deliver glucagon to the injection site.

Since the patient is continuously subject to the infusion, the pH of the formulation should be in the pH range of 4-8 to avoid site discomfort. Commercially available formulations of glucagon are only intended for a single rescue dose of 1 mg and therefore are prepared at a very low pH of approximately 2. These formulations come in a kit containing a lyophilized glucagon powder and diluent in a separate bottle. These must be combined before use and immediately administered, and according to the label, any excess is to be thrown away. This is because the glucagon is not stable once reconstituted. General observations of pure glucagon powder in solution at pH 4 show that it gels within 2 days. Glucagon from a commercially available rescue kit at pH 2 does not gel, but it does chemically degrade over time and drops to less than 90% potency in three days. Adjustment of this formulation to pH 4 hastened the decomposition by precipitation (FIG. 4). A formulation of glucagon (referred to as BIOD 901) has been designed to be soluble at neutral pH, and have less tendency to gel at 37° C. This was accomplished by combining a solubilizing agent lyso-myristoyl-phosphocholine (1-myristoyl-2-hydroxy-sn-glycero-3-phosphocholine), a simple sugar glucose, and a preservative m-cresol. BIOD 901 contains 1 mg/mL glucagon+2 mg/ml LMPC+45 mg/mL glucose+2 mg/mL m-cresol, and is made from a basic solution which is adjusted to pH 7.

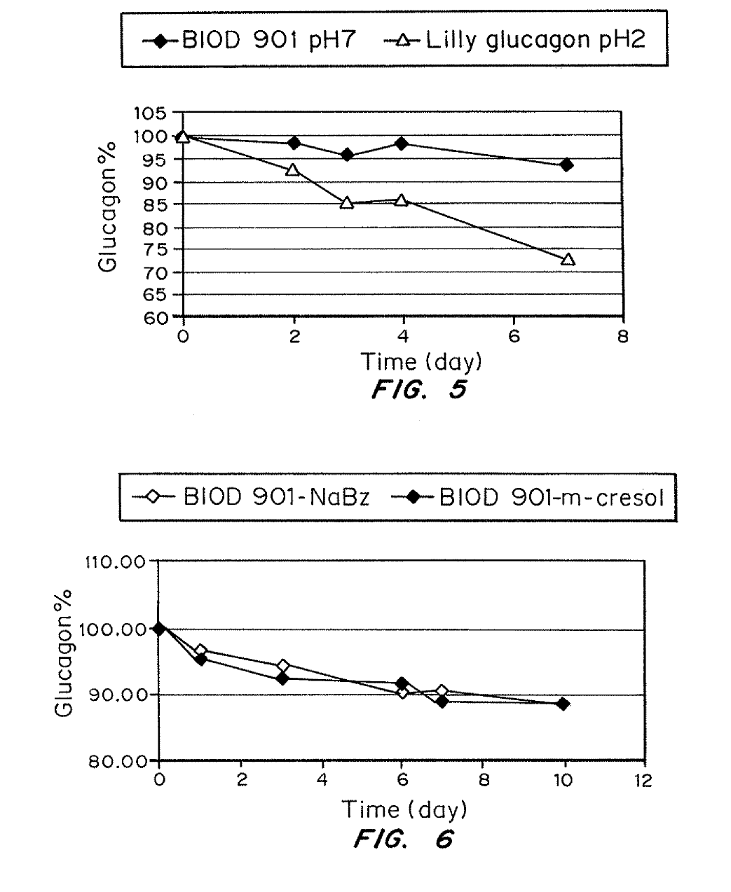

Results of stability testing under accelerated conditions (37° C.) of BIOD 901 and the commercial Lilly preparation at pH 2 which is adjusted to pH 4 are shown in FIGS. 4 and 5. The pH adjustment to 4 was done in an effort to reduce the acidity of the formulation, making it more suitable for pumping into the near neutral pH subcutaneous tissue. The Lilly glucagon at pH 2 dropped to 85% by day 3, while BIOD 901 was well above 90% on day 7. The Lilly glucagon pH 4 precipitated out of solution on the first day. The BIOD 901 began to gel at ten days, well beyond the required 7 days. A control formulation without glucose gelled at approximately 6 days, demonstrating the stabilizing effect of the glucose. This new glucagon formulation made with a combination of LMPC, glucose and preservative was significantly more stable compared to glucagon with LMPC or glucose alone (FIGS. 2 and 3).

### Example 4

### Comparison of Alternative Preservatives: m-Cresol and Sodium Benzoate

Due to incompatibilities with some plastic storage containers, an alternative to m-cresol was tested. Using a combination of 2 mg/mL LMPC, 45 mg/ml glucose and 1 mg/ml glucagon, two preservatives, m-cresol and sodium benzoate 0.5 mg/mL were tested. Glucagon powder was first dissolved into the lipid solution at a concentration of 2 mg/mL glucagon and 4 mg/mL of lipid. Concentrated glucose, m-cresol/sodium benzoate solution were then added to the solution and briefly mixed. The expected final concentrations are in Table 1. The solution pH was adjusted to about 7 and samples were placed in a 37° C. chamber. Samples were filtered through a 2 μm filter and analyzed by HPLC and remaining glucagon (as a function of time) of the two formulations were graphed in FIG. 6.

TABLE 1
Compositions of test glucagon formulations

| Glucagon | lipid | Sugar | Preservatives |
|---|---|---|---|
| 1 mg/mL | 2 mg/mL lyso myristoyl | 45 mg/ml | 2 mg/mL |
| | phosphocholine (LMPC) | glucose | m-cresol |
| 1 mg/mL | 2 mg/mL LMPC | 45 mg/ml | 2 mg/mL |
| | | glucose | Sodium benzoate |

The results (FIG. 6) show the average glucagon remaining was essentially the same with either sodium benzoate or m-cresol.

### Example 5

### Addition of Phosphate Buffer to Fix pH at 7.3 in Glucagon Formulation

Further refinement of the formulation included the addition of phosphate buffer. The new formulation, BIOD 902, contains 1 mg/mL glucagon, 2 mg/mL LMPC, 2 mg/mL m-cresol, 45 mg/ml glucose and 5 mM phosphate buffer, pH 7.3 (does not require pH adjustment). This was prepared at neutral pH (no glucagon exposure to basic environment.

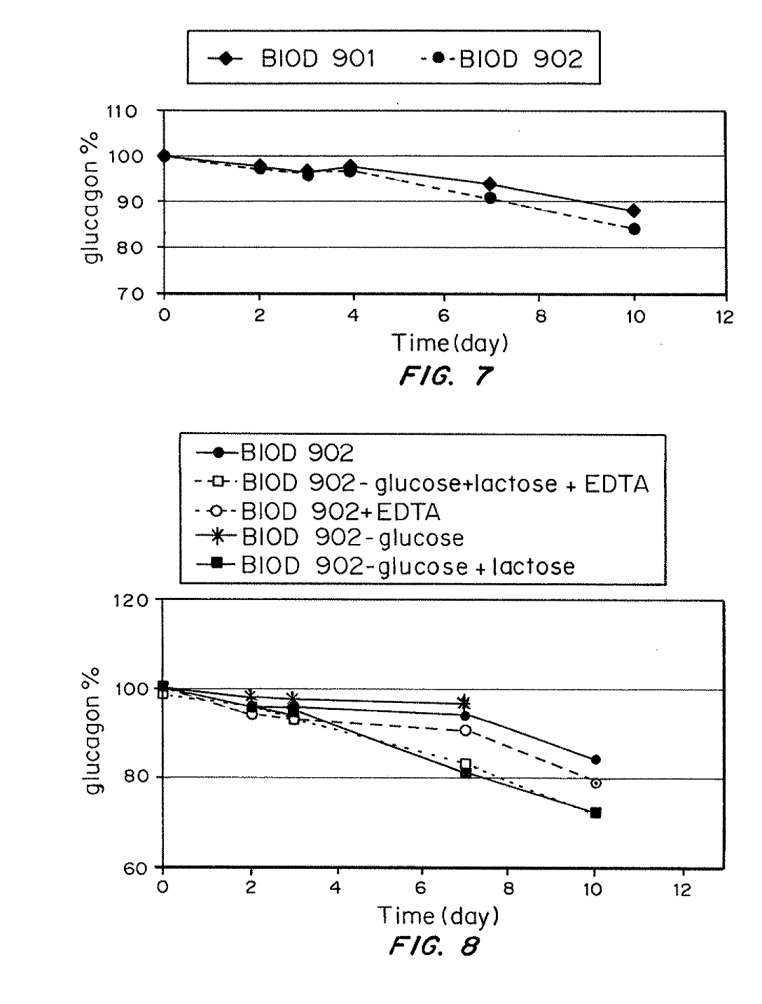

Stability of BIOD 902 was comparable to BIOD 901. However, a commercial formulation would benefit from a controlled pH (FIG. 7).

### Example 6

### Stability of BIOD 902 Following Addition of EDTA, Lactose and Blends of EDTA and Lactose Compared to Glucose

Further studies were performed on BIOD 902 with formulation variations to evaluate potential stability benefits.

Test formulations included either:

Addition of EDTA (0.25 mg/ml) (with and without glucose)
Addition of Lactose (90 mg/ml) (no glucose)
Addition of Lactose (90 mg/mL) and EDTA (0.25 mg/mL, no glucose)

BIOD 902 and BIOD 902 without glucose (BIOD-glucose) initially have the best stability, until the gelation occurred with BIOD 902-glucose after day 7. Addition of EDTA did not show an improvement to BIOD 902. The addition of lactose and EDTA plus lactose (both without glucose) showed no benefit and were chemically degraded by day 7. BIOD 902 (which contains glucose) had lost 10% of its potency by day 10, but it was still in solution and did not show any signs of gelation. This is an important benefit to use with pump devices.

### Example 7

### In Vivo Glucose Response to Glucagon Administration in Diabetic Miniature Swine

Five diabetic miniature swine were fed a full breakfast on the morning of the study and given a prandial insulin with their food. Three hours later, additional insulin was given intravenously to lower the glucose to 50-100 mg/dL prior to glucagon dosing. Glucose was monitored every 10 minutes via the strip method to determine the appropriate time (when glucose levels had dropped to 50-100 mg/dL) for subcutaneous dosing of the 50 μL of glucagon (1 mg/mL solution). The glucagon formulation BIOD 901 pH 7.2 was compared to the commercially prepared Lilly formulation pH 2 in the same swine. In addition, the BIOD 901 formulation was incubated for 3 days at 37 C, and given to the same swine the following day. This was done to confirm activity in vivo after experiencing stressed conditions.

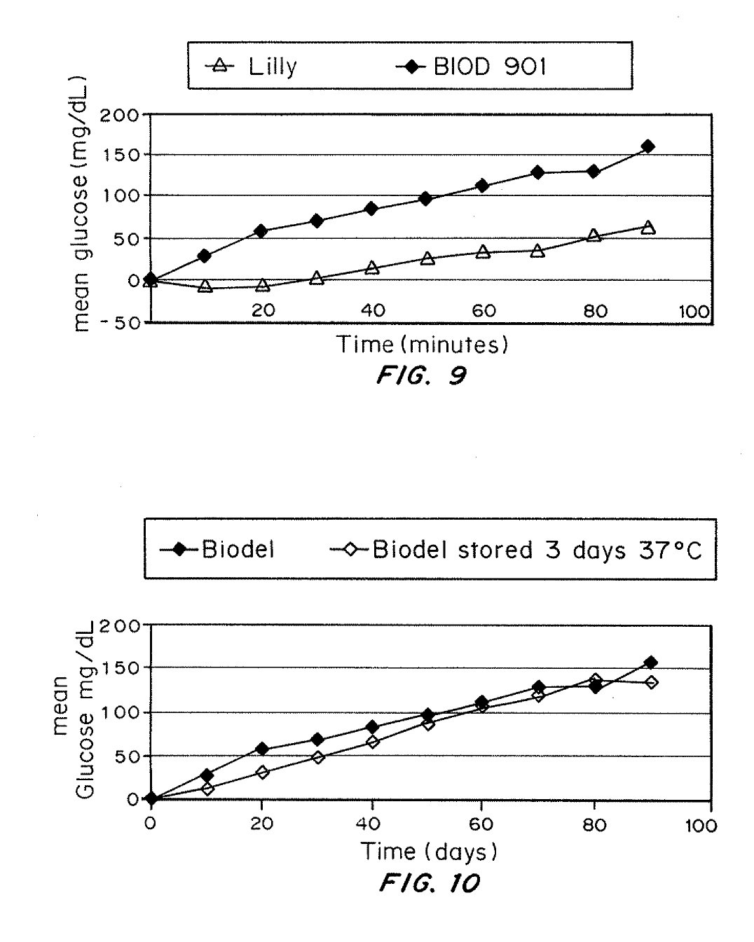

FIG. 9 shows the mean increase from baseline of blood glucose over time (post glucagon administration) to the same 5 swine given either BIOD 901 or Lilly glucagon freshly prepared. The pigs responded well to the glucagon, elevating their glucose levels considerably post injection. The BIOD 901 formulation appears to elevate blood glucose faster than the commercial formulation (Lilly). FIG. 10 is a graph of the mean increase from baseline of blood glucose over time of BIOD 901 before and after 3 days at 37° C. The almost superimposable results show that there was little change in efficacy of the glucagon after incubation at 37° C.

Modifications and variations of the present invention will be obvious to those skilled in the art from the foregoing detailed description and are intended to come within the scope of the following claims. The teachings of all references cited herein are specifically incorporated by reference.

We claim:

1. A stabilized glucagon formulation comprising
Glucagon,
A surfactant, and
A mono or disaccharide,
Wherein the surfactant and saccharide are in an effective amount to stabilize the glucagon, and
Wherein the osmolarity is approximately 250 to 310 mOs and the pH 4-7.5.

2. The formulation of claim 1 wherein the surfactant is a lysophospholipid, phospholipid, glycerophospholipid or amphilic block copolymer.

3. The formulation of claim 2 wherein the surfactant is myristoyl lysophosphocholine

4. The formulation of claim 1 wherein the sugar is a monosaccharide or diasaccharide with an alkyl chain length ranged from C8 to C12.

5. The formulation of claim 4 wherein the sugar is selected from the group consisting of lactose, maltose and glucose.

6. The formulation of claim 1 further comprising a preservative.

7. The formulation of claim 1 wherein the concentration range for the glucagon is between 0.5 and 5 mg/mL; sugar is between 20 and 100 mg/mL; and surfactant is between 0.1 and 10 mg/mL.

8. The formulation of claim 7 wherein the concentration range for the glucagon is between 0.8 and 1.5 mg/mL; sugar is between 36 and 72 mg/mL, and surfactant is between 0.5 and 5 mg/mL.

9. The formulation of claim 1 comprising a preservative in a concentration of between 0.2 and 3 mg/mL.

10. The formulation of claim 1 comprising a microemulsion or liposome.

11. The formulation of claim 1 comprising a reconstitutable powder.

12. The formulation of claim 11 wherein the glucagon is provided in a two vial kit with one vial containing glucagon and a second vial containing diluent, wherein the glucagon is reconstituted with diluent immediately before use.

13. The formulation of claim 1 wherein the glucagon is administered in a pump.

14. The formulation of claim 1 wherein the glucagon is provided in a single vial as a solution.

15. The formulation of claim 1 wherein the pH of the glucagon is in the physiological range.

16. The formulation of claim 1 wherein the pH of the glucagon is in the acidic range.

17. A method of making a stable glucagon solution comprising providing the formulation of claim 1.

18. A method of treating a patient in need thereof comprising administering the glucagon of claim 1.
